(19) United States
(12) Patent Application Publication
(10) Pub. No.: US 2004/0235016 A1
(43) Pub. Date: Nov. 25, 2004

(54) Nanocylinder-modified surfaces

(75) Inventors: Robert J. Hamers, Madison, WI (US); Sarah Baker, Madison, WI (US); Tami Lasseter, Madison, WI (US)

Correspondence Address: FOLEY & LARDNER, 150 EAST GILMAN STREET, P.O. BOX 1497, MADISON, WI, 53701-1497, US

(73) Assignee: Wisconsin Alumni Research Foundation

(21) Appl. No.: 10/773,753

(22) Filed: Feb. 6, 2004

Related U.S. Application Data
(60) Provisional application No. 60/445,611, filed on Feb. 7, 2003.

(51) Int. Cl. C12Q 1/68; G01N 33/53; G01N 33/537; G01N 33/543; C12M 1/34

(52) U.S. Cl. 435/6; 435/7.92; 435/287.2

(57) ABSTRACT

This invention provides surfaces having nanocylinders, such as carbon nanotubes, attached thereto through biomolecular interactions, devices made from assemblies of nanocylinder-modified surfaces, and methods for producing nanocylinder modified surfaces. A variety of biomolecular interactions may be used to attach the nanocylinders to the surfaces, including hybridization of complementary oligonucleotide sequences and receptor-ligand interactions.

### CROSS-REFERENCE TO RELATED APPLICATION

This application claims the benefit of U.S. provisional patent application No. 60/445,611, filed Feb. 7, 2003, the entire disclosure of which is incorporated herein by reference and for all purposes.

### STATEMENT OF GOVERNMENT RIGHTS

Research funding was provided for this invention by the National Science Foundation under Grant Number CHE 0071385, the National Institute for Health under Grant Number 8 R01 EB00269-02 and the Department of Defense under Grant Number F30602-01-2-0555. The federal government has certain rights in this invention.

### FIELD OF THE INVENTION

This invention relates to surfaces modified with nanocylinders through biomolecular interactions, assemblies made from nanocylinder-modified surfaces, and methods for producing nanocylinder-modified surfaces.

### BACKGROUND OF THE INVENTION

Recently there has been a tremendous interest in the use of carbon nanotubes and related nano-sized objects in electronic devices, field emission sources, and chemical sensors. The reason for the recent interest stems from the fact that carbon nanotubes are characterized by their strength (they are stronger than steel), high thermal and electrical conductivity, and biocompatibility with a variety of biomolecules. These features make carbon nanotubes well suited for a vast array of commercial applications, including nanoelectronic circuits.

Presently, nanotubes can be prepared through batch processing or by catalytic deposition. Both methods yield a mixture of metallic and semiconducting tubes, with specific properties varying from tube to tube depending on the individual diameters and chirality. The use of nanotubes in many applications is highly dependent on having reproducible electrical properties. For example, in the fabrication of nanotube-based transistors it is important to control whether the tubes are metallic or semiconducting. At the present time, nanotubes are either grown in place and then tested individually for the desired electronic properties, or else they are deposited and those having undesired properties are removed selectively by applying a voltage across the tubes. These methods suffer from the disadvantage that they take a considerable amount of time and are therefore not well suited for mass production. At the same time, the biotechnology industry has developed the ability to specifically pattern surfaces with a wide range of biomolecules. These “bio chips” are typically used for genetic screening.

Additionally, interest has recently developed in the use of adducts of nanotubes with biomolecules in biosensing applications and as a possible means of implementing nanoscale assembly, using the selectivity of biomolecular interactions to control assembly of nanometer-sized objects. Previous studies have focused primarily on the use of non-covalent interaction. Unfortunately, non-covalent functionalization, which typically involves coating a nanotube with various large molecules or polymers, may disrupt the nanotube's structure over a substantial length of the nanotube, which may have a significant effect on the electrical and chemical properties of the nanotube.

### SUMMARY OF THE INVENTION

The present invention provides surfaces that are modified with nanocylinders through biomolecular interactions, nanocylinder assemblies and devices held together through biomolecular interactions, and methods for making the same.

The term nanocylinder, as used herein, is defined to refer to both nanotubes and nanorods. The term nanocylinder is further defined to include other nanometer-sized objects having a generally well-defined cylindrical (i.e. rod-like or tube-like) geometry but which differ from nanorods and nanotubes in their aspect ratios (typically these other nanocylinders are longer and often narrower than nanorods). For example, the term nanocylinder also refers to nanowires, nanofiliments, and nanowhiskers. The use of the term nanocylinder is not intended to imply that the rod-like nanometer-sized object must have a circular cross-section, other cross-sectional shapes are suitable.

As the name implies, nanocylinders are characterized in that they have a nanometer-sized cross-sectional dimension, and often a nanometer-sized length dimension as well. For example, some nanocylinders have a diameter of one micrometer or less. The nanocylinders may be made a variety of materials, including, but not limited to, carbon, gold, and silver. As one of skill in the art will recognize, the choice of appropriate nanocylinders will depend in large part on the intended application.

One aspect of the present invention provides a surface having one or more nanocylinders attached thereto through biomolecular interactions between one or more biomolecules bound to the surface and one or more complementary biomolecules bound to the nanocylinders. The resulting assemblies are useful in a range of applications, such as electronic devices, including sensors and nanoelectronic circuits. In the assemblies of the present invention the biomolecules play at least two roles; first they serve to provide the controlled attachment of the nanocylinders to the surface, and second, in some instances, the biomolecules increase the solubility of the nanocylinders in solvents, such as organic solvents. The second role is significant because the low width to length ratio of nanocylinders provides them with low solubility in most solvents, which has hampered previous attempts to use nanocylinders, such as nanotubes and nanorods, in nanoscale assembly and distinguishes nanocylinders from other nano-sized objects, such as nanospheres, nanocrystals, and the like, which are easily dissolved in most solvents.

In certain embodiments, the biomolecules are covalently linked to the nanocylinder(s). This is advantageous because covalent linkages make the nanocylinder-biomolecule adducts chemically and thermally stable, and because selective modification at a few specific locations may minimize the disruption of the structure and electronic properties of the nanocylinders.

One embodiment of a nanocylinder-modified surface includes (a) a substrate having a surface, the surface having at least one biomolecule bound thereto; and (b) a nanocylinder having at least one complementary biomolecule covalently linked thereto, wherein the nanocylinder is attached to the substrate surface through biomolecular interactions between the at least one biomolecule on the substrate surface and the at least one complementary biomolecule on the nanocylinder.

One important advantage to this approach to assembling nanocylinders on surfaces is that both the location and alignment of the nanocylinders on a surface can be controlled by the selective placement of the biomolecules and their complementary biomolecule partners on the surface and the nanocylinders, respectively. The degree of control may be enhanced by using complementary biomolecule pairs that undergo specific binding to ensure that a given biomolecule linked at a certain location on a nanocylinder will bind only to its complementary biomolecule at a predetermined location on a surface. The ability to control the placement of nanocylinders on a substrate allows for the production of patterned surfaces where the nanocylinders are laid out relative to one another in a predetermined design. The patterned surfaces are useful for many applications, including nanoelectronic circuits. In addition, the controlled assembly of nanocylinders on surfaces allows for the production of a variety of electronic devices and sensors, including devices constructed from assemblies of one or more nanocylinders and one or more surfaces bound by biomolecular interactions between complementary biomolecule pairs.

Bioswitches and nanocylinder bridges are two examples of nanocylinder assemblies that may be produced in accordance with the present invention.

One embodiment of a bioswitch that acts as a biomolecular sensor for detecting the presence of an analyte may be constructed from two electrodes and a nanocylinder, such as a nanotube. Specifically, the bioswitch includes: (a) a first electrode having at least one biomolecule bound thereto; (b) a second electrode having at least one biomolecule bound thereto, wherein the first and second electrodes are separated by a gap; (c) a nanocylinder having at least two biomolecules bound thereto; and (d) a detector connected to the first and second electrodes for measuring the impedance between the first and second electrodes. In this configuration, the at least one biomolecule bound to the first electrode and one of the at least two biomolecules bound to the nanocylinder are capable of binding the analyte between them and the at least one biomolecule bound to the second electrode and the other of the at least two biomolecules bound to the nanocylinder are capable of binding the analyte between them, such that the nanocylinder bridges the gap between the first and second electrodes and modifies the electrical impedance (i.e. resistance, capacitance, or inductance, or a combination thereof) between the first and second electrodes.

In this embodiment, the biomolecule(s) on the substrate surface, the biomolecules on the nanocylinder, and the analyte should be selected such that the presence of the nanostructure in contact with or very near the surfaces after the connections are formed between the electrodes changes the AC conductivitiy (i.e. the AC impedance) of the system. This configuration acts as a switch. In the absence of analyte the system will have a first impedance, however, once the analyte is exposed to the system, it binds between the biomolecules on the electrodes and the nanocylinder, changing the impedance of the system. The closing of the switch may be detected by measuring the change in impedance that occurs in the presence of the analyte. In this embodiment, each junction between the electrode and the nanocylinder essentially forms a capacitor. Thus, the entire switch is essentially two capacitors in series, linked by a conductive wire.

Another embodiment of the invention provides a nanobridge connecting two surfaces. Presently, such bridges, which are typically made from carbon nanotubes, are constructed by growing nanotubes directly on a surface. However, this process is inefficient and does not always guarantee a bridge will be formed. The nanobridge of the present invention includes: (a) a first surface having at least one biomolecule bound thereto; (b) a second surface having at least one biomolecule bound thereto; and (c) a nanocylinder having at least two biomolecules bound thereto, wherein one of the at least two biomolecules on the nanocylinder is bound to the at least one biomolecule on the first surface and the other of the at least two biomolecules on the nanocylinder is bound to the at least one biomolecule on the second surface to form a bridge between the first and the second surfaces.

In fabricating nanobridges, it is advantageous (but not necessary) for one of the at least two biomolecules on the nanocylinder to specifically bind to the biomolecule bound to the first surface, but not to the biomolecule bound to the second surface, and for the other of the at least two biomolecules on the nanocylinder specifically to bind to the biomolecule bound to the second surface, but not to the biomolecule bound to the first surface. This construction ensures that the nanocylinder will bridge the two surfaces, rather than binding only to one surface or the other.

Nanotubes and nanorods are examples of nanocylinders that are well suited for use in the present invention. Carbon nanotubes are a specific example of nanotubes that may be used advantageously due to their strength and thermal and electrical conductivities. Carbon nanotubes are well known and are commercially available, these nanotubes (sometimes called buckytubes) are long, cylindrical carbon structures consisting of hexagonal graphite molecules attached at the edges. Metal nanorods, including, but not limited to, silver and gold nanorods, are also useful due to their thermal and electrical conductivities. In addition, metal nanorods may be produced with internal structures that allow them to be selectively functionalized at selected locations with different biomolecules.

DNA molecules, or other oligonucleotides, such as RNA molecules, are an example of biomolecules that may be bound to surfaces and nanocylinders in accordance with the present invention. In this design oligonucleotides on a nanocylinder have nucleotide sequences that are complementary to and capable of hybridizing with oligonucleotides on a surface. The use of complementary oligonucleotide pairs as binding partners allows the user to control the location and alignment of the nanocylinders on a surface by taking advantage of the selectivity and reversibility of the hybridization and provides the ability to design, fabricate, and link different oligonucleotides to a variety of different surfaces and nanoscale objects.

Receptors and their corresponding ligands are other examples of biomolecules that may be bound to surfaces and nanocylinders in accordance with the present invention. In this system the biomolecular interaction that attaches the nanocylinder to the surface is a ligand-receptor interaction. One specific example of a receptor-ligand pair that may be used with the present invention is the biotin-avidin (or biotin-Streptavidin) pair. In this design, biotin molecules may be covalently linked to a nanocylinder and avidin (or Streptavidin) molecules may be bound, typically through another biotin molecule, to a surface. The protein-ligand binding that occurs when the biotin is exposed to the avidin (or Streptavidin) is strong and leads to the irreversible binding of the nanocylinder to the surface.

Another aspect of the invention provides a method of selectively assembling nanocylinders on surfaces to produce nanocylinder-modified surfaces, such as those described above. This method may be carried out by exposing a biomolecularly functionalized surface, of the type described above, to one or more nanocylinders that are themselves bound to one or more biomolecules capable of binding to the biomolecules on the substrate surface, such that the biomolecules on the surface and the complementary biomolecules on the nanocylinders attach the nanocylinders to the substrate surface through biomolecular interactions.

Further objects, features and advantages of the invention will be apparent from the following detailed description when taken in conjunction with the accompanying drawings.

### BRIEF DESCRIPTION OF THE DRAWINGS

In the drawings:

FIG. 1 is a schematic illustration of a chemical scheme for producing covalently-modified adducts of single-walled carbon nanotubes (SWNTs) with DNA (1e) and with biotin (1f).

FIG. 2 shows fluorescence images (black=high intensity) of DNA-SWNT adducts that were hybridized with complementary and 4-base mismatched sequences, as described in the Examples below. The top row shows the initial hybridization. The second row shows the same samples after denaturing in urea, and the bottom row shows the same samples after hybridizing a second time with a different sequence, as described in the Examples below.

FIG. 3 shows the biologically-directed assembly on SWNTs on a surface. The white and grey images respectively represent red and green fluorescence intensity using a 605-nm long-pass filter and a 512-nm bandpass filter, respectively. Two samples were used; one glass surface (center images) was modified only with biotin and rhodamine-labeled avidin, while the second (right images) was modified with biotin, then rhodamine-labeled avidin, and then immersed in a solution of biotin-modified nanotubes that were also labeled with green fluorescein dye. Each sample was modified with biotin in two circular regions. The “red” (shown as white) and “green” (shown as grey) images were obtained simultaneously for each sample.

FIG. 4 shows an illustration of a bioswitch that uses a receptor-ligand interactions to assemble a nanotube across a pair of electrodes.

FIG. 5 shows an illustration of a bioswitch that uses oligonucleotide hybridization to assemble a nanotube across a pair of electrodes.

FIG. 6 shows an image of a gold nanowire connected across two gold electrodes using avidin-biotin interactions.

FIG. 7 shows a graph of the current across two gold electrodes before and after a gold nanowire is connected between them.

### DETAILED DESCRIPTION OF THE PREFERRED EMBODIMENTS

The present invention provides surfaces modified with nanocylinders, electronic devices and sensors made from nanocylinder-modified surfaces, and methods of producing nanocylinder-modified surfaces.

The nanocylinder-modified surfaces are made from one or more nanocylinders bound to one or more surfaces through biomolecular interactions between biomolecules bound to the surface(s) and complementary biomolecules bound to the nanocylinder(s). The arrangement of the nanocylinder(s) on the surface(s) may be controlled by the selective placement of the biomolecules on the nanocylinder(s) and the surface(s) and by the specificity of the biomolecular interactions between the biomolecules on the surface(s) and those on the nanocylinder(s). This design provides control and flexibility in the arrangement of nanocylinders on surfaces, making the nanocylinder-modified surfaces useful for a broad range of applications.

In certain embodiments, the biomolecules bound to the nanocylinders are bound by covalent linkages. The use of covalent bonding to anchor the biomolecules to the nanocylinders produces a nanocylinder-biomolecule adduct that is chemically and thermally stable, and accessible. In addition, the use of covalent linkages between the biomolecules and the nanocylinder localizes any structural disruptions to the attachment sites which reduces the effects of the functionalization on the electronic properties of the nanocylinder. This is supported by a report showing that oxidation of “defect-free” HipCO nanotubes (Carbon Nanotechnologies, Inc.) retained the van Hove features, thereby indicating that the electronic properties are relatively unperturbed by formation of oxidized surface sites. See J. Am. Chem. Soc., 124, 12418-12419 (2002).

One important area where the nanocylinder-modified substrates of the present invention may be applied is in nanoelectric circuits where the nanocylinders must be appropriately aligned on a substrate. Electrically conducting and semiconducting nanotubes and nanorods are well-suited for use as the nanocylinders in these nanoelectric circuits. Carbon nanotubes, also known as buckytubes, are an example of nanotubes that may be advantageously used to modify a surface. Carbon nanotubes are characterized by high strength and high thermal and electrical conductivity. These structures are well known in the art and are typically produced through high pressure carbon monoxide (HipCO) processes, pulsed laser vaporization, or arc discharge processes. Carbon nanotubes may be single-walled nanotubes (SWNTs) or multiple-walled nanotubes (MWNTs). Both types are suitable for use in the present invention. The carbon nanotubes may be either metallic or semiconducting, depending upon the diameter and chirality of the nanotube.

Nanorods are another group of nanocylinders that are well suited for use with the present invention. Like the nanotubes, the nanorods may be semiconducting or conducting nanorods. Nanorods include nanorods made from semiconducting materials such as silicon and indium phosphide. Nanorods further include metal nanorods including, but not limited to, nanorods made from gold and/or silver. Other suitable metal nanorods may be made from iron, cobalt, platinum, palladium, molybdenum and copper. Metal nanorods have the advantage that a metal nanorod can be constructed of two different materials (i.e. a first metal and a second metal), such as silver and gold. The resulting nanorod will include at least one region of the first metal and at least one region of the second metal and the at least two regions may be selectively functionalized. For example, the metals may be chosen such that one metal undergoes functionalization under a given set of reaction conditions and the other metal does not. Alternatively, the first and second metals may be selected such that they undergo different functionalization reactions, thereby providing different biomolecular functionalities on the first and second regions.

In accordance with one embodiment of this invention, a nanocylinder is attached to a surface through biomolecular interactions between a biomolecule bound to the surface and a complementary biomolecule covalently linked to the nanocylinder. The biomolecule bound to the surface may be bound through one or more covalent or non-covalent linkages, or a combination thereof. For example, the biomolecule may be bound to the surface by non-covalent interactions with a linking group or molecule, which is itself covalently linked to the surface.

The biomolecule or biomolecules may be bound to a nanocylinder along the periphery and/or at the end of the structure. However, the number of biomolecules bound to the nanocylinders and the chemistry used to produce covalent linkages on the nanocylinders should be chosen such that the effects on the structure and electrical properties of the nanocylinders is minimized. Carbon nanotubes are frequently characterized by the presence of carboxylic acid groups at their open tip ends and on structural defects along their periphery. Thus, when carbon nanotubes are used as the nanocylinders, biomolecules may be attached to the tip ends and/or to structural defects by derivatizing the tip ends and coupling the derivatized tip ends to the biomolecules. Because carboxylic acid groups may be derivatized by a variety of well-known reactions, it is possible to functionalize the tip ends with a variety of biomolecules. One method for fuictionalizing carbon nanotubes with biomolecules is described in Nature, 394, 52-55 (1998) which is incorporated herein by reference. Other exemplary methods for covalently functionalizing carbon nanotubes with biomolecules are presented in the Examples section below.

A nanocylinder may be modified with one or more of the same biomolecule or may be selectively modified with two or more different biomolecules each having a different complementary biomolecule to which it binds with specificity. In the latter design, the placement and orientation of the nanocylinder on a surface or between surfaces can be controlled by the location of each member of a specific binding pair on the nanocylinder and the surface.

The ability to control the location, alignment, and/or the orientation of one or more nanocylinders on a surface allows the user to produce patterned surfaces wherein the nanocylinders are arranged in designs that are predetermined by the placement and specificity of the complementary biomolecule pairs on the surface and the nanocylinders. Such patterned surfaces are particularly valuable in the area of nanoelectronic circuits.

In addition to creating patterned surfaces, the controlled assembly of nanocylinders can be used to create assemblies and devices made by attaching one or more nanocylinders, to one or more surfaces through biomolecular interactions. For example, as discussed in greater detail below, selective modification of a nanotube may be used to create a bridge between two surfaces.

The biomolecules used to functionalize a nanocylinder may include any biomolecule that may be bound to the nanocylinder without losing its ability to bind to its complementary biomolecule on the surface. Similarly, the biomolecules used to functionalize the surface may include any biomolecule that may be bound to that surface without losing its ability to bind to its complementary biomolecule on the nanocylinder. As used herein, the term “complementary biomolecules” covers any biomolecule pair that is capable of binding together. The binding between the complementary biomolecule pair may be specific, semi-specific, or non-specific. However, in many applications complementary biomolecule pairs that undergo specific or semi-specific binding are preferred because they allow for more flexibility and control in the placement, orientation, and alignment of the nanocylinders on and between surfaces. The biomolecules may have a single binding site through which they interact with a complementary biomolecule or they may have multiple binding sites through which they interact with one or more complementary biomolecules.

Biomolecules and complementary biomolecules for use in the present invention are well-known in the art. Suitable biomolecules and complementary biomolecules include, but are not limited to, biomolecules independently selected from the group consisting of oligonucleotide sequences, including both DNA and RNA sequences, amino acid sequences, proteins, protein fragments, ligands, receptors, receptor fragments, antibodies, antibody fragments, antigens, antigen fragments, enzymes and enzyme fragments. Thus, the biomolecular interactions between the complementary biomolecule pairs include, but are not limited to, receptor-ligand interactions (including protein-ligand interactions), hybridization between complementary oligonucleotide sequences (e.g. DNA-DNA interactions or DNA-RNA interactions), and antibody-antigen interactions.

In one exemplary embodiment of the invention the biomolecule bound to the substrate surface is a protein and the complementary biomolecule covalently linked to the nanocylinder is a ligand capable of specifically binding with the protein. For example, the protein may be avidin or Streptavidin and the ligand may be biotin. The interaction of biotin with avidin has one of the largest known binding constants (10¹⁵ M⁻¹). This large binding constant makes the biotin-avidin interaction useful for the fabrication of robust nanoscale structures.

The surface to which the nanocylinders are attached may be an insulating surface, a semiconducting surface, or a conducting surface, depending on the intended application for the system. Suitable examples of insulating surfaces include, but are not limited to, glass surfaces. Suitable examples of semiconducting surfaces include, but are not limited to, silicon surfaces. Suitable examples conducting surfaces include, but are not limited to, metal surfaces (such as gold or silver surfaces), glassy carbon surfaces, and diamond thin film surfaces.

The nanocylinder-modified surfaces may be incorporated in assemblies to provide various electronic devices and sensors. Two such devices, a bioswitch and a nanobridge, are described in detail below.

A biomolecular sensor, or “bioswitch”, may be made from the following components: (a) a first electrode having at least one biomolecule bound thereto; (b) a second electrode having at least one biomolecule bound thereto, wherein the first and second electrodes are separated by a gap; (c) a nanocylinder having at least two biomolecules bound thereto; and (d) a detector connected to the first and second electrodes for measuring the inductance between the first and second electrodes. In this configuration, a biomolecule bound to the first electrode and one of the biomolecules bound to the nanocylinder bind an analyte between them to form a first connection. Similarly, a biomolecule bound to the second electrode and one of the biomolecules bound to nanocylinder bind an analyte between them to form a second connection, wherein the nanocylinder bridges the gap between the first and second electrodes and completes an electrical connection between the first and second electrodes and further wherein the presence of the nanocylinder attached in close proximity to electrodes the produces a measurable change in the inductance of the system.

In some embodiments the first and second electrodes are functionalized with the same biomolecules and in others the first and second electrodes are each functionalized with a different biomolecule.

Conducting or semiconducting nanotubes and nanorods, and carbon nanotubes in particular, are examples of nanocylinders that may be used in the biosensor of this invention. Nanocylinders are useful, because they may be very long (in some cases one hundred, two hundred, or even more microns in length) which allows the electrodes themselves to be made with dimensions much smaller (e.g. less than about 10 microns in length) than the nanocylinders. Standard lithography techniques are well known for producing electrodes with such small dimensions. This helps to ensure that the nanocylinders will bridge across the two electrodes, rather than just attaching to one or the other, when the two electrodes are functionalized with the same biomolecule. In this design, the nanocylinder has twice the binding energy by virtue of being able to interact with twice as many biomolecules.

In one embodiment the biosensor may be used to sense the presence of a protein analyte using receptor-ligand interactions. In this design, ligands capable of binding to the protein analyte of interest are bound to the nanocylinder(s) and the electrodes. The chosen analyte is a protein capable of simultaneously binding between a ligand on the nanocylinder and a ligand on an electrode to form a connection between the nanocylinder and the electrode. The ligands on the electrodes and the nanocylinder may be the same or different depending on the number and type of binding sites available on the protein analyte.

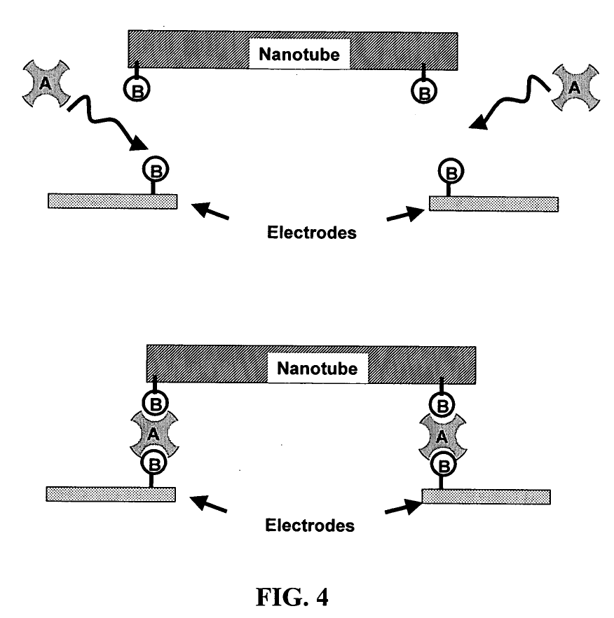

One illustrative example of such a sensor may be made by binding biotin ligands to the two electrodes and the nanocylinder. This configuration is capable of detecting the presence of avidin (or Streptavidin) in a given sample because avidin (or Streptavidin) has four binding sites for biotin and, as such, is capable of forming a connection between the nanocylinder(s) and the electrodes by simultaneously binding to the biotin molecules on both. As shown in FIG. 4, in this embodiment the presence of analyte or target molecule “A” (such as avidin) is being sensed. A surface with two electrodes is modified with a complementary molecule “B” (such as biotin). Carbon nanotubes are also modified with the complementary molecule “B”. The presence of a target molecule that will bind to “B” molecules on the surface and on the nanotubes provides a connection between the surface and the nanotube. The target molecule “A” must have at least two binding sites in order to link the nanotubes and the surface. The molecule avidin is known to have four binding sites and therefore meets this criterion.

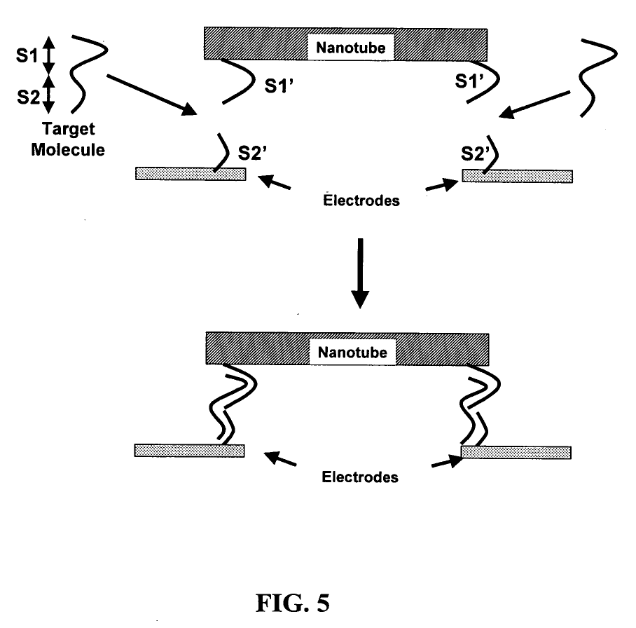

FIG. 5 shows another illustrative example where oligonucleotide hybridization is used to produce a bioswitch. In this embodiment, a target DNA oligonucleotide is being sensed. The target molecule has a specific sequence of bases, which can be thought of as two partial sequences S1 and S2. S1 and S2 can be continguous, but this is not necessary. DNA oligonucleotides having sequence S1′, where S1′ is the sequence complementary to S1, can be bonded to the carbon nanotubes. DNA olignucleotides having the sequence S2′, where S2′ is the sequence complementary to S2, can be bonded to the surface to two electrodes. When the target molecule is present, it will bind to both S1′ and S2′, thereby linking the nanotubes to the electrodes.

In another embodiment, a nanocylinder may be used as a bridge between two surfaces, particularly two metal surfaces. An example of such a bridge includes: (a) a first surface having at least one biomolecule bound thereto; (b) a second surface having at least one biomolecule bound thereto; and (c) a nanocylinder having at least two biomolecules bound thereto, wherein one of the biomolecules on the nanocylinder is bound to a biomolecule on the first surface and the other biomolecule on the nanocylinder is bound to the a biomolecule on the second surface to form a bridge linking the first and the second surfaces.

The use of nanocylinders is advantageous because a biomolecule may be conveniently covalently linked at or near the each end of the nanocylinder. In some embodiments, the bridge may optionally be designed such that one of the at least two biomolecules on the nanocylinder specifically binds to a biomolecule on the first surface, but not to a biomolecule on second surface, and the other biomolecule on the nanocylinder specifically binds to a biomolecule on the second surface, but not to a biomolecule on the first surface. In this construction a nanotube, or other nanocylinder, may be modified with a different biomolecule on or near each of its two ends. A first surface is modified with a biomolecule that is complementary to the biomolecule at one end of the nanotube and a second surface is modified with a biomolecule that is complementary to the biomolecule at the other end of the nanotube. When the selectively modified nanotube and the two surfaces are allowed to interact, the nanotube forms a bridge between the two surfaces attached at either end by specific complementary biomolecular interactions.

Another aspect of the invention provides a method of selectively assembling nanocylinders on surfaces to produce nanocylinder-modified surfaces and assemblies, such as those described above. This method may be carried out by exposing a biomolecularly functionalized surface, of the type described above, to one or more nanocylinders that are themselves functionalized with one or more complementary biomolecules, such that the biomolecules on the surface and the complementary biomolecules on the nanocylinders attach the nanocylinders to the surface through biomolecular interactions. This method provides a simple process that may be carried out at room temperature. In applications where the biomolecular interactions are weak or where there is a risk that the biomolecules may denature, the connection between the nanocylinder and the surface may be further strengthened by annealing the surface having the nanocylinder arranged thereon at a temperature sufficient to strengthen the attachment of the nanocylinder to the surface.

### EXAMPLES

### Example 1

### DNA-Modified Single-Walled Carbon Nanotubes

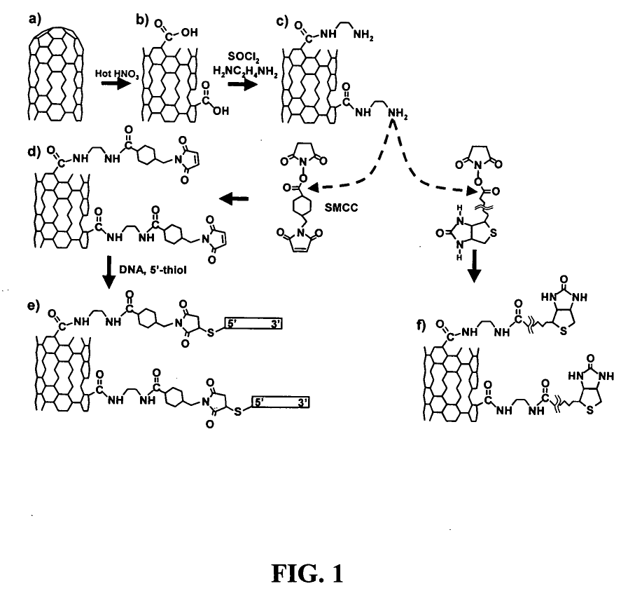

Experiments were performed using two different sources of single-walled carbon nanotubes. Single-walled carbon nanotubes (SWNTs) (Carbolex, Lexington, Ky.) were first purified by refluxing the as-received nanotubes in 3 M nitric acid for 24 hours (FIG. 1, steps a and b) and then washing the SWNTs with water using a 0.6 micron polycarbonate membrane filter (Millipore). HipCO Tubes (Carbon Nanotechnologies, Inc., Houston, Tex.) were also prepared by oxidation in 9:1 H₂SO₄:30% H₂O₂ solution. To functionalize the nanotubes with amine groups, the purified, oxidized material (˜60% of initial weight of SWNTs) was dried under vacuum and then suspended in 1 ml of anhydrous dimethylformamide (DMF) in an ultrasonic bath. This dispersion was immediately added to 20 ml thionyl chloride (Aldrich) and heated under reflux for 24 hours to convert the carboxylic acids to acyl chlorides. These nanotubes were rinsed over a 0.2 micron PTFE membrane (Millipore) with anhydrous THF to remove excess SOCl₂, and were then added to ethylene diamine (neat, Aldrich) and stirred for 3-5 days in order to form the amine-terminated product depicted in FIG. 1c.

The amine-terminated nanotubes (FIG. 1c) provide a versatile starting point for further modification. To prepare DNA-modified SWNTs, the tubes were reacted with the heterobifunctional cross-linker succinimidyl 4-(N-maleimidomethyl)cyclohexane-1-carboxylate, (SMCC), leaving the surface terminated with maleimide groups (FIG. 1d) which were then reacted with thiol-terminated DNA to produce DNA-modified SWNTs (FIG. 1e). Alternatively, the amine-terminated SWNTs can be reacted with N-hydroxy succinimidyl biotin (Vector Labs), producing SWNTs covalently linked to biotin as depicted in FIG. 1f.

Several different DNA oligonucleotides were used in these experiments. To optimize the DNA-SWNT linkage chemistry, a 32-base oligonucleotide (5′-HS-C₆H₁₂-T₁₅GC TTA ACG AGC AAT CGT FAM-3′) (“S1”) was used. This oligonucleotide was modified at the 5′ end using the reagent 5′-thiol modifier C6 (Glen Research, Sterling, Va.) to give a thiol group for attachment to the maleimide group on the nanotubes (FIG. 1d), and was modified at the 3′ end using 6-FAM amidite (Applied Biosystems, Foster City, Calif.) to attach a fluorescein group.

Tests to verify the formation and stability of the covalent linkage between the nanotubes and the DNA were performed by directly linking DNA molecules with a fluorescent tag. These tests showed that the DNA-SWNT adducts are quite stable even in the presence of hot surfactant-containing solutions that would normally denature physically-adsorbed molecules. This, together with detailed chemical information presented elsewhere in Nano. Lett., 2, 1413-1417 (2002), which is incorporated herein by reference, establishes that the DNA molecules are indeed covalently linked to the SWNTs.

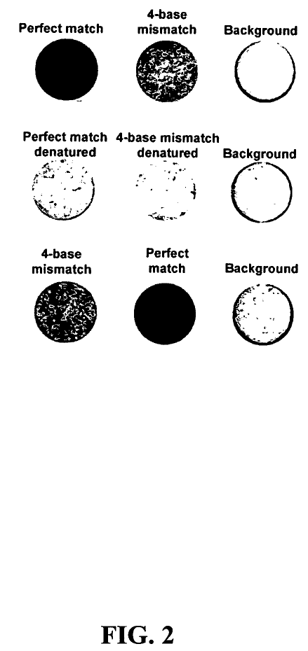

Since the above experiment proved that the DNA-SWNT adducts are stable, further experiments were conducted to test whether the DNA molecules that are tethered to the SWNTs remain biochemically accessible to hybridization, and whether the attachment to the nanotubes significantly impacts the selectivity for hybridization with complementary vs. non-complementary sequences. For these experiments, DNA without a fluorescent tag was linked to the nanotubes, and the hybridization of these DNA-SWNT adducts with fluorescently-tagged complementary and non-complementary sequences of DNA in solution was investigated. These experiments were conducted using the oligonucleotide “S2”, with the sequence (5′-HS-C₆H₁₂-T₁₅GC TTA ACG AGC AAT CG -3′), linked to the nanotubes. After immobilization onto the SWNTs following the procedures above, the resulting DNA-nanotube adduct was then portioned into two aliquots, and each was immersed in a 5 micromolar solution of DNA oligonucleotides that were labeled at the 5′ end with fluorescein. The first sequence, “S3”, (5′-FAM-CG ATT GCT CGT TAA GC-3′), has sixteen bases complementary to S2. The second sequence, “S4”, consists of the 16-base sequence (5′-FAM-CG TTT GCA CGT TTA CC-3′) that has four-base mismatch to S2. Each sample was hybridized for 2 hours at 37° C. with shaking, washed using a 0.2 micron polycarbonate membrane with SDS/2×SSPE buffer, and then placed in a 96 well microtiter plate in buffer. FIG. 2 shows the resulting fluorescence image of this experiment. The top row shows the fluorescence images (black=high intensity; white=low or no intensity) for hybridization of S2-SWNT with its complement, S3 (left) and with the 4-base mismatch, S4 (middle). The image at right shows the background from an empty titerplate well. Measurement of the fluorescence intensity within each well yields a median value of 1287 I.U. for the perfect match (left), 680 I.U. for the mismatch (middle) and 427 I.U. for the background. Since there is a much higher intensity from the perfect-matched pair (S2−SWNT+S3) than the mismatched pair (S2−SWNT+S4), we conclude that hybridization of the DNA-SWNT adducts with solution-phase oligonucleotides is highly specific.

The reversibility of hybridization was tested by denaturing with 8.3 M urea solution, and then re-hybridizing to a different sequence. After denaturing, the fluorescence images (FIG. 2, middle row) show only low levels of fluorescence from the two samples (intensity=304 I.U. from perfect match, 267 I.U. from 4-base mismatch) comparable to the background level (intensity=238 I.U.). These denatured samples were then hybridized a second time. In this second hybridization, the sample that was previously hybridized with a perfect match was now hybridized with a mismatched sequence, and vice versa. The images in the bottom row of FIG. 2 show that again, the fluorescence intensity of the 4-base mismatched pair S2-SWNT+S4 (bottom left, intensity=441 I.U.) is close to that of the background (bottom right, 257 I.U.), while the relative intensity of the perfect mach S2-SWNT+S3 (bottom middle, intensity=1073 I.U.) is much higher than either. Again, the hybridization appears to be quite specific.

The above results strongly point to the successful synthesis of covalently-linked DNA-SWNT adducts. These experiments show that the DNA-SWNT adducts are biochemically accessible and exhibit a high degree of selectivity in hybridization experiments. This high degree of selectivity can be potentially useful in a number of applications, such as fabrication of nanoscale chemical sensors and in the use of biological molecules to direct the assembly of nanotubes and other nanoscale objects.

### Example 2

### Biotin-Modified Single-Walled Carbon Nanotubes and Substrates Modified with Same

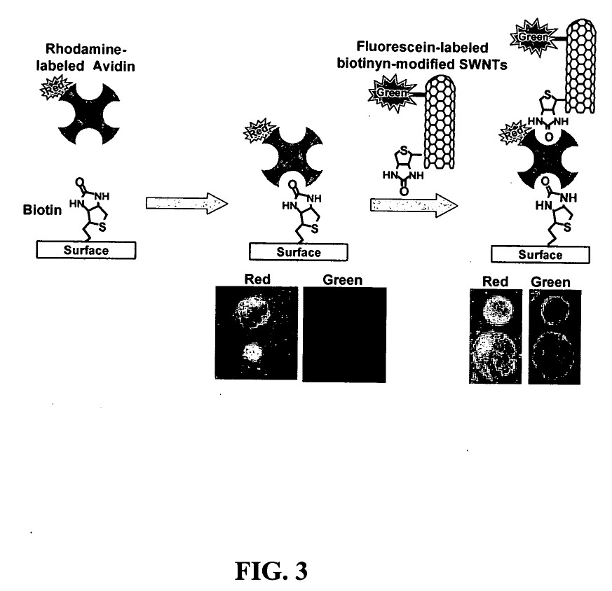

While DNA hybridization involves weak interactions, the interaction between biotin (a small vitamin) and avidin (a small protein) is one of the strongest biomolecular interactions known, with a formation constant of 10¹⁵ M⁻¹. This very high stability implies that the biotin-avidin interaction can be used to assist in the assembly of nanoscale supramolecular architectures by making use of the fact that avidin has four sites that can bind to biotin molecules. In this example, the biotin-avidin interaction was used to selectively link biotin-modified SWNTs to biotin-modified surfaces, using avidin as a kind of glue to bind the assembly together. This experiment involves multiple steps, as shown schematically in FIG. 3.

Biotin-modified SWNTs were produced using chemistry very similar to that used for preparing DNA-modified nanotubes. The procedure involves fabrication of amine-terminated SWNTs and then reacting these with a small molecule containing a biotin group and an N-hydroxy succinimide group, which forms a covalent link to the amine groups to produce a covalently-linked SWNT-biotin adduct like that shown in FIG. 1f.

A second method of preparing biotin-modified carbon nanotubes may also be used. In this method, carbon nanotubes are first oxidized in an acid solution (3:1 H₂SO₄: HNO₃) for one hour while sonicating. This oxidation step is necessary to produce initial sites for further functionalization to occur. The nanotubes are then filtered and rinsed through with water to remove excess acid. A suspension with the nanotubes, EDC (1-ethyl-3-[3-dimethylaminopropyl]-carbodiimide hydrochloride 50 mM in DMF) and NHS (N-hydroxysuccinimide 100 mM in DMF) is made and allowed to react for 1.5 hr. The nanotubes are then rinsed with excess DMF to remove unreacted EDC and NHS. This step results in activated carboxyl nanotubes which will readily react with amines under slightly basic conditions. Amine-terminated biotin (5-(Biotinamido)pentylamine) and amine terminated fluorescein (aminoacetamido fluorescein), in equimolar amounts, are then added to the nanotubes (suspended in a pH 8.0 solution) for 2 hours. A final filtration and rinsing step removes all excess reagents and results in biotin functionalized carbon nanotubes.

Because proteins such as avidin are often sensitive and easily subject to denaturation or other degradation processes, avidin was linked to the surfaces via a two-step procedure in which surfaces of silicon, glassy carbon, or glass were first modified to provide accessible primary amine groups. These amine-terminated surfaces were then reacted with a modified N-hydroxy-succinimide (NHS) ester of biotin, yielding the covalent biotin-SWNT adduct depicted in FIG. 1f. Silicon, glassy carbon, and glass were selected as substrate surfaces because they can all be modified via similar chemistry to amine groups as described in J. Am. Chem. Soc., 122, 1205-1209 (2000), which is incorporated herein by reference, while having significantly different optical and electrical properties. Data presented here was obtained on amine-terminated glass surfaces that were purchased commercially (GAPS-II, Coming, Coming, N.Y.). The second step, linking biotin to the amine-terminated surfaces, can also be performed using several different reagents. The present experiments used Sulfo-Succinimidyl-6-(biotinamido) hexanoate from Pierce Endogen. However, a number of compounds are available commercially with NHS esters linked to biotin; these compounds differ slightly but would be expected to provide similar functionality. Details of this linkage have been eliminated from FIG. 1f to improve the clarity.

FIG. 3 shows the procedure, along with the fluroescence data. Corning GAPS-II amine-terminated glass surfaces were modified with biotin. Avidin that was fluorescently labeled with rhodamine dye was then bonded to the surface, thereby producing an avidin-terminated surface that fluoresced in the red region of the spectrum. The rhodamine dye is labeled as “red” in FIG. 3. Carbon nanotubes were covalently linked to biotin as in FIG. 1f, and were simultaneously linked to the green fluorescent dye fluorescein using an NHS-ester of fluorescein from Molecular Probes, Eugene, Oreg. The fluorescein dye is labeled as “green” in FIG. 3. Covalently linking the nanotubes simultaneously to biotin and fluorescein provides a way of directly imaging the nanotubes via fluorescence in the green region of the spectrum. The avidin-modified glass surfaces where then briefly dipped into a dilute solution of nanotubes (modified with biotin and fluorescein, as described above) and then rinsed with a standard buffer solution.

FIG. 3 (lower panels) shows the resulting images of fluorescence intensity, measured at two different wavelengths, along with a control experiment from an avidin-modified sample that was not exposed to nanotubes. In FIG. 3, the images labeled “red” show the fluorescence intensity, which appears white in the images, obtained using a 605 nm long pass filter, representing fluorescence from the rhodamine-labeled avidin molecules covalently linked to the glass surface. The images labeled “green” show the fluorescence intensity, which appears grey in the images, measured using a 512 nm band pass filter, which represents fluorescence from the fluorescein groups covalently linked to the nanotubes. A control experiment (center) shows that the avidin-modified surface fluoresces in the red, but no fluorescence is observed in the green on the avidin-modified surface before being exposed to the nanotubes. After being exposed to biotin, the fluorescence images at right show fluorescence both in the red (from the avidin) and in the green (from the nanotubes). It is important to note that the fluorescence from the rhodamine-labeled avidin and the fluorescein-labeled nanotubes is only observed in the surface regions that were modified with biotin (two spots). Other regions of the surface do not show significant fluorescence intensity.

These images therefore show that biotin-modified SWNTs will link specifically to surface regions that have been modified with avidin. This experiment establishes that it is possible to use the biotin-avidin interaction as a means of controlling the assembly of nanotubes onto a surface. The use of biomolecular interactions (including, but not limited to, protein-substrate interactions, antibody-antigen interactions, or DNA hybridization) between a surface-bound biomolecule and a biologically-modified nanotube is expected to be a general method that can be used to achieve biomolecularly-assisted assembly of nanotubes.

The integration of nanotubes with biological molecules provides a wealth of opportunities in nanoscale assembly, by using the highly selective nature of biochemical interactions to control the behavior of nanoscale objects. The results above show that it is possible to prepare covalently-linked adducts of single-walled nanotubes with DNA and with biotin. The use of DNA hybridization provides a potential pathway for controlling complex objects by taking advantage of the high degree of selectivity and reversibility, and the ability to readily design, synthesize, and link different DNA sequences to a variety of surfaces and nanoscale objects. The use of biotin and avidin provides complementary qualities, since the very high binding constant of avidin-biotin leads to nearly irreversible binding. Example 3

### DNA-Modified Metal Nanorods

Methods for the production of nanorods are well known in the art. Descriptions of these methods may be found in Science, 294, 137-140 (2001); JACS, 124, 4020-4026 (2002); and the Journal of Materials Chemistry, 7, 1075-1087 (1997), each of which is incorporated herein by reference. Briefly, nanorods of varying lengths and compositions can be prepared using electrochemical reduction in a template such as nanoporous alumina. In this process, a porous alumina membrane (other materials can also be used) is first coated with metal on one side. A plating solution is applied to the opposite side and is used to form an electrochemical cell in which the metal ions are reduced to free metal in the pores of the membrane. The use of sequential deposition reactions of different metals has been demonstrated to produce metal “barcodes”, as described in Science Vol. 294, pp. 137-140 (2001). A metal nanorod consisting of two different metals (“A” and “B”) could be selectively functionalized with different molecules in different regions. For example, if a nanorod consisting of gold at the ends and silver in the center was exposed to a solution consisting of alkanethiol with an amine or carboxylic acid group at the end, this would lead the nanorod to be selectively functionalized at the gold locations and not at the silver locations, due to the high affinity of alkanethiols for gold.

Functionalization of the gold surface or surface regions of a nanotube is accomplished using methods analogous to those used on conventional gold substrates. For example, an amine-functionalized gold nanorod can be made according to the procedure described in Langmuir, 16, 2192-2197 (2000), which is herein incorporated by reference. Briefly, functionalization of the gold regions of the nanorods is accomplished by immersing the rods in a solution of 11-mercaptoundecylamine, 1 millimolar in ethanol, to produce an amine-modified nanorod. This step is identical to published work on planar gold surfaces. (Langmuir, vol. 16, pp. 2192-2197 (2000)). The amine-terminated nanorods can then be linked to DNA via an additional two steps that have been widely used on a number of different amine-terminated planar surfaces (see, for example, Nature Materials, 1, 253-257 (2002), and Langmuir, 18, 788-796 (2002), both of which are incorporated herein by reference) and on amine-modified carbon nanotubes (see Nano Letters, 2, 1413-1417 (2002), which is incorporated herein by reference). The nanorods are then exposed to a 1.5 mM solution of the heterobifunctional cross-linker sulfosuccinimidyl-4-(N-maleimidomethyl)cyclohexane-1-carboxylate (SSMCC) in triethanolamine buffer solution (pH 7) for about 20 minutes. The NHS-ester group in this molecule reacts specifically with the —NH₂ groups of the surface to form an amide bond. The maleimide moiety can then reacted with thiol-modified DNA (250 μM thiol DNA in 0.1M pH 7 TEA buffer) by placing the DNA directly onto the surface in a humid chamber and allowing it to react for >6 hrs at room temperature.

### Example 4

### Gold Nanowire Switch

A nanoswitch made from a gold nanowire attached across two gold electrodes was produced. Using standard ultraviolet lithography, gold electrodes made from a 40 nm layer of gold on a 10 nm layer of titanium were fabricated on an oxidized silicon wafer. The gold electrodes were then exposed to an ultraviolet lamp (254 nm) for 15 minutes. This generated ozone which removed any residual organic matter from the surface. The surface was then rinsed with deionized water and ethanol. The sample was then immersed in 1 millimolar (mM) MUAM (11-amino-1-undecaethiol hydrochloride) (Dojindo, Gaitherburg, Md.) in an ethanol solution to grow a compact self assembled monolayer. After about 24 hours, the electrodes were rinsed with deionized water and a small drop (e.g. about 20 microliters) of 1 mM sulfosuccinimidyl-6-(biotinamido) hexanoate (SSBAH) solution (pH 7.0) (Pierce Chemical, Rockford, Ill.) was placed on the electrodes. After about 30 minutes the electrodes were rinsed with deionized water to get rid of any extra SSBAH. This provided gold electrodes functionalized by biotin groups.

The biotin functionalized gold electrodes were then further functionalized with avidin by dripping about 20 μL of 1 mg/mL avidin (Vector Laboratories, Burlingame, Calif.) in HEPES (N-[2-hydroxyethyl] piperazine-N′-[2-ethanesulfonic acid]) buffer (pH 8.0) (Sigma, St. Louis, Mo.) onto the biotin-modified electrodes. The electrodes were refrigerated at 4° C. for about 30 minutes then rinsed with deionized water. Finally, the electrodes were rinsed twice with 0.1% Triton-X-100 (Fisher Scientific) in 2×SSPE buffer (Promega, Madison, Wis.) for about 30 minutes.

Gold nanowires (approximately 200 nm in diameter and 7 μm in length) were obtained using electrochemical deposition of a gold plating solution (Alpha Aesar, Ward Hill, Mass.) on an alumina membrane. The gold nanowires were then functionalized by immersing them in 1 millimolar (mM) MUAM in an ethanol solution to grow a compact self assembled monolayer, rinsing them with deionized water and contacting them with a small drop of 1 mM SSBAH solution (pH 7.0), using the same procedure used to functionalize the gold electrodes. This provided gold nanowires functionalized by biotin groups.

To form a switch between the gold electrodes, a dilute suspension of biotin-modified gold nanowires was dripped onto the biotin/avidin functionalized electrodes. In some cases, it may be advantageous to refrigerate the electrodes and rinse them with deionized water and/or 0.1% Triton-X-100 SSPE solution to remove any non-specifically bonded nanowires.

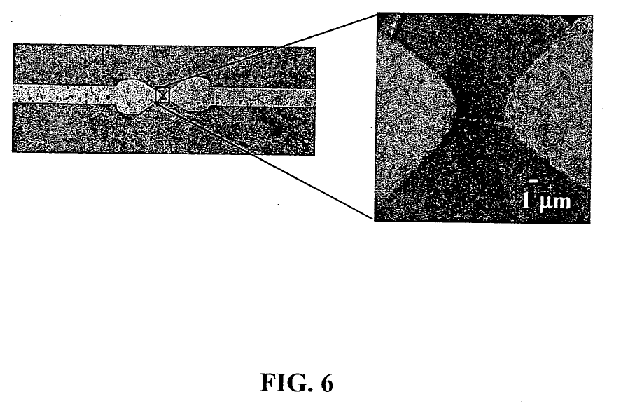

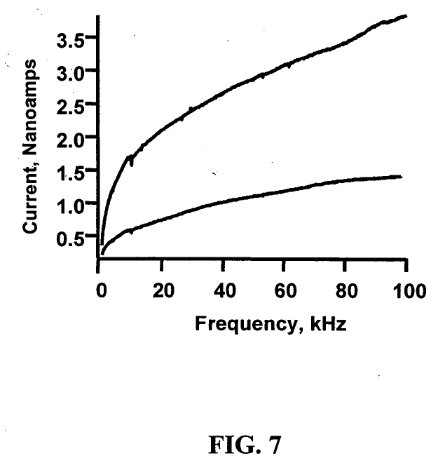

FIG. 6 shows an image of a gold nanowire connected across the two gold electrodes. Measurements of the current across the electrodes were made before and after the formation of the nanowire switch. Electrical measurements can be made in a number of ways. Here, measurements were made using a standard function generator to generate a sinusoidal waveform (up to 100 mV amplitude, frequencies of 0-200 kHz), and measuring the in-phase and out-of-phase components of the current using a lock-in amplifier. Electrical measurements were made using an AC voltage of 10 millivolts. The results, shown in FIG. 7, clearly show an increase in current in the presence of the nanowires.

It is understood that the invention is not confined to the particular embodiments set forth herein, but embraces all such forms thereof as come within the scope of the following claims.

What is claimed is:

1. A modified substrate comprising:
(a) a substrate having a surface, the surface having at least one biomolecule bound thereto; and
(b) at least one nanocylinder having at least one complementary biomolecule covalently linked thereto;
wherein the at least one nanocylinder is attached to the surface through biomolecular interactions between the at least one biomolecule on the surface and the at least one complementary biomolecule on the at least one nanocylinder.

2. The modified substrate of claim 1 wherein the at least one nanocylinder is a nanotube or nanorod.

3. The modified substrate of claim 1 wherein the at least one nanocylinder is a carbon nanotube.

4. The modified substrate of claim 1 wherein the at least one nanocylinder is a gold or silver nanorod.

5. The modified substrate of claim 1 wherein the at least one biomolecule bound to the surface and the at least one complementary biomolecule covalently linked to the at least one nanocylinder are independently selected from the group consisting of oligonucleotide sequences, amino acid sequences, proteins, protein fragments, ligands, receptors, receptor fragments, antibodies, antibody fragments, antigens, antigen fragments, enzymes, and enzyme fragments.

6. The modified substrate of claim 1 wherein the at least one biomolecule bound to the surface comprises an oligonucleotide sequence and the at least one complementary biomolecule covalently linked to the at least one nanocylinder comprises a complementary oligonucleotide sequence.

7. The modified substrate of claim 1 wherein the at least one biomolecule bound to the surface and the at least one complementary biomolecule covalently linked to the at least one nanocylinder form a protein-ligand pair.

8. The modified substrate of claim 7 wherein the at least one biomolecule bound to the surface comprises avidin or Streptavidin and the at least one complementary biomolecule covalently linked to the at least one nanocylinder comprises biotin.

9. The modified substrate of claim 1 wherein the substrate is selected from the group consisting of silicon, glass, glassy carbon, gold, and diamond thin film substrates.

10. The modified substrate of claim 1 wherein the covalent linkage comprises the reaction product of an amine terminated nanocylinder with a molecule comprising a maleimide group.

11. The modified substrate of claim 10 wherein the covalent linkage further comprises the reaction product of the molecule comprising the maleimide group and a thiol terminated biomolecule.

12. A method of selectively arranging nanoscale objects on a substrate comprising exposing a substrate having a surface, the surface having at least one biomolecule bound thereto, to at least one nanocylinder having at least one complementary biomolecule covalently linked thereto, wherein biomolecular interactions between the at least one biomolecule bound to the surface and the at least one complementary biomolecule covalently linked to the at least one nanocylinder attach the at least one nanocylinder to the surface.

13. The method of claim 12, further comprising annealing the surface having the at least one nanocylinder attached thereto at a temperature sufficient to strengthen the attachment between the surface and the at least one nanocylinder.

14. The method of claim 12 wherein the method is carried out at room temperature.

15. The method of claim 12 wherein the at least one nanocylinder is a nanotube or nanorod.

16. The method of claim 12 wherein the at least one nanocylinder is a carbon nanotube.

17. The method of claim 12 wherein the at least one nanocylinder is a gold or silver nanorod.

18. The method of claim 12 wherein the at least one biomolecule bound to the surface and the at least one complementary biomolecule covalently linked to the at least one nanocylinder are independently selected from the group consisting of oligonucleotide sequences, amino acid sequences, proteins, protein fragments, ligands, receptors, receptor fragments, antibodies, antibody fragments, antigens, antigen fragments, enzymes, and enzyme fragments.

19. The method of claim 12 wherein the at least one biomolecule bound to the surface comprises an oligonucleotide sequence and the at least one complementary biomolecule covalently linked to the at least one nanocylinder comprises a complementary oligonucleotide sequence.

20. The method of claim 12 wherein the at least one biomolecule bound to the surface and the at least one complementary biomolecule covalently linked to the at least one nanocylinder form a protein-ligand pair.

21. The method of claim 20 wherein the at least one biomolecule bound to the surface comprises avidin or Streptavidin and the at least one complementary biomolecule covalently linked to the at least one nanocylinder comprises biotin.

22. The method of claim 12 wherein the substrate is selected from the group consisting of silicon, glass, glassy carbon, gold, and diamond thin film substrates.

23. The method of claim 12 wherein the covalent linkage comprises the reaction product of an amine terminated nanocylinder with a molecule comprising a maleimide group.

24. The modified substrate of claim 23 wherein the covalent linkage further comprises the reaction product of the molecule comprising the maleimide group and a thiol terminated biomolecule.

25. A biomolecular sensor for sensing the presence of an analyte, the sensor comprising:
(a) a first electrode having at least one biomolecule bound thereto;
(b) a second electrode having at least one biomolecule bound thereto, wherein the first and second electrodes are separated by a gap;
(c) at least one nanocylinder having at least two biomolecules bound thereto; and
(d) a detector connected to the first and second electrodes for measuring the impedance between the first and second electrodes; wherein the at least one biomolecule bound to the first electrode and one of the at least two biomolecules bound to the at least one nanocylinder are capable of binding the analyte between them, and further wherein the at least one biomolecule bound to the second electrode and one of the at least two biomolecules bound to the at least one nanocylinder are capable of binding the analyte between them, wherein the at least one nanocylinder bridges the gap between the first and second electrodes and further wherein the close proximity of the nanocylinder to the electrodes produces a measurable impedance change.

26. The biomolecular sensor of claim 25 wherein the at least one nanocylinder is a nanotube or nanorod.

27. The biomolecular sensor of claim 25 wherein the at least one nanocylinder is a carbon nanotube.

28. The biomolecular sensor of claim 25 wherein the at least one nanocylinder is a gold or silver nanorod.

29. The biomolecular sensor of claim 25 wherein the at least one biomolecule bound to each of the electrodes, the at least two biomolecules bound to the at least one nanocylinder, and the analyte are independently selected from the group consisting of oligonucleotide sequences, amino acid sequences, proteins, protein fragments, ligands, receptors, receptor fragments, antibodies, antibody fragments, antigens, antigen fragments, enzymes, and enzyme fragments.

30. The biomolecular sensor of claim 25 wherein the analyte comprises a protein and the at least one biomolecule bound to the first electrode, the at least one biomolecule bound to the second electrode, and the at least two biomolecules bound to the at least one nanocylinder comprise ligands capable of binding to the analyte.

31. The biomolecular sensor of claim 25 wherein the analyte comprises avidin or Streptavidin and the at least one biomolecule bound to the first electrode, the at least one biomolecule bound to the second electrode, and the at least two biomolecules bound to the at least one nanocylinder comprise biotin.

32. A nanocylinder bridge comprising:
(a) a first surface having at least one biomolecule bound thereto;
(b) a second surface having at least one biomolecule bound thereto; and
(c) a nanocylinder having at least two biomolecules bound thereto, wherein one of the at least two biomolecules on the nanocylinder is bound to the at least one biomolecule on the first surface and the other of the at least two biomolecules on to the nanocylinder is bound to the at least one biomolecule on the second surface to form a bridge between the first and the second surfaces

33. The nanocylinder bridge of claim 32 wherein the nanocylinder is a carbon nanotube.

34. The nanocylinder bridge of claim 32 wherein each of the at least two biomolecules covalently linked to the carbon nanotube is linked to or near a different end of the carbon nanotube.

35. The nanocylinder bridge of claim 32 wherein one of the at least two biomolecules covalently linked to the nanocylinder specifically binds to the biomolecule bound to the first surface, but not to the biomolecule bound to the second surface, and the other of the at least two biomolecules covalently linked to the nanocylinder specifically binds to the biomolecule bound to the second surface, but not to the biomolecule bound to the first surface.

36. The nanocylinder bridge of claim 32 wherein the first and second surfaces are metal surfaces.

37. A patterned surface comprising a surface having a plurality of nanocylinders arranged thereon in a predetermined pattern, wherein the nanocylinders are attached to the surface by biomolecular interactions between biomolecules bound to the surface and their complementary biomolecules bound to the nanocylinder, and further wherein the pattern is predetermined by the locations of the biomolecules on the surface and their complementary biomolecules on the nanocylinders.
